(19) United States
(12) Patent Application Publication
(10) Pub. No.: US 2007/0129652 A1
(43) Pub. Date: Jun. 7, 2007

(54) Methods and apparatus for intracranial ultrasound therapies

(76) Inventor: Henry Nita, Redwood Shores, CA (US)

Correspondence Address: Raymond Sun, 12420 Woodhall Way, Tustin, CA, 92782, US

(21) Appl. No.: 11/274,356

(22) Filed: Nov. 15, 2005

(51) Int. Cl. A61H 1/00; A61B 8/00; A61H 1/02

(52) U.S. Cl. 601/2; 600/454; 600/439

(57) ABSTRACT

A method and apparatus for delivering ultrasound energy to a patient's intracranial space involves forming at least one hole in the patient's skull, positioning an access device around the hole, advancing at least one ultrasound delivery device partially through the access device, and transmitting ultrasound energy from the ultrasound delivery device.

### RELATED APPLICATION AND INCORPORATION BY REFERENCE

Applicant expressly incorporates herein by this reference the entire disclosures in pending application Ser. No. 11/165,872, filed on Jun. 24, 2005, and pending application Ser. No. 11/203,738, filed on Aug. 15, 2005.

### BACKGROUND OF THE INVENTION

1. Field of the Invention

The present invention relates generally to medical methods and apparatus. More specifically, the invention relates to methods and apparatus for intracranial ultrasound delivery, which may include diagnostic ultrasound, therapeutic ultrasound, or both, delivered through a hole in the skull.

2. Background Art

Stroke is characterized by the sudden loss of circulation to an area of the brain, resulting in a corresponding loss of neurologic function. Also called cerebrovascular accident or stroke syndrome, stroke is a nonspecific term encompassing a heterogeneous group of pathophysiologic causes, including thrombosis, embolism, and hemorrhage. Strokes currently are classified as either hemorrhagic or ischemic. Acute ischemic stroke refers to strokes caused by thrombosis or embolism and accounts for 80% of all strokes.

More than 400,000 people per year in the U.S. have a first-time stroke. At current trends, this number is projected to increase to one million per year by the year 2050. Stroke is the third leading cause of death and the leading cause of disability in the U.S. Worldwide, cerebrovascular disease was the second leading cause of death in 1990, killing over 4.3 million people. Cerebrovascular disease was also the fifth leading cause of lost productivity, as measured by disability-adjusted life years (DALYs). In 1990, cerebrovascular disease caused 38.5 million DALYs throughout the world. And although stroke often is considered a disease of the elderly, 25% of strokes occur in persons younger than 65 years. When the direct costs (care and treatment) and the indirect costs (lost productivity) of strokes are considered together, strokes cost the American society $43.3 billion per year.

Until very recently, almost nothing could be done to help patients with acute stroke. Little treatment existed for ischemic stroke until 1995, when the National Institute of Neurologic Disorders and Stroke (NINDS) recombinant tissue-type plasminogen activator (rt-PA) stroke study group first reported that the early administration of rt-PA benefited some carefully selected patients with acute ischemic stroke. Encouraged by this breakthrough study and the subsequent approval of t-PA for use in acute ischemic stroke by the U.S. Food and Drug Administration, administration of t-PA has become increasingly more prevalent in stroke treatment. Treating patients early enough in the course of stroke, however, is an extremely challenging hurdle to effective treatment of stroke. Furthermore, t-PA for stroke treatment is much more effective if delivered locally at the site of blood vessel blockage, but such delivery requires a great deal of skill and training, which only a small handful of medical professionals possess.

One proposed enhancement for treatment of stroke is the administration of trans-cranial Doppler (TCD) at high frequencies (i.e., approximately 2 MHz) and low intensities, which is normally used for diagnostic functions. TCD has been shown not only to be effective in visualizing clots, but also to be effective in lysing clots in the middle cerebral arteries, in combination with lytic drugs such as t-PA and/or microbubbles. TCD has also been shown to be safe, with no clinically significant brain bleeding effects. (See, for example: A. V. Alexandrov et al., “Ultrasound-Enhanced Thrombolysis for Acute Ischemic Stroke,” N. Engl. J. Med. 351;21, Nov. 18, 2004; and W. C. Culp and T. C McCowan, “Ultrasound Augmented Thrombolysis,” Current Medical Imaging Reviews, 2005, 1, 5-12.) The primary challenge in using TCD to enhance stroke treatment, however, is that the skull attenuates the ultrasound signal to such a high degree that it is very difficult to deliver high-frequency, low-intensity signals through the skull. Using higher intensity ultrasound signals, in an attempt to better penetrate the skull, often causes unwanted bleeding of small intracranial blood vessels and/or heating and sometimes burning of the scalp. The only other option is to carefully aim a high-frequency, low-intensity TCD signal through a small window in the temporal bone of the skull to arrive at the middle cerebral artery, which is the technique described in the studies cited above and is the only technique studied thus far.

There are two main drawbacks to delivering high-frequency TCD through the temporal window. First, such delivery requires a high level of skill, and only a small handful of highly trained ultrasonographers are currently capable of performing this technique. Second, not all intracranial blood vessels are reachable with TCD via the temporal window. For example, although the temporal window approach may work well for addressing the middle cerebral artery, it may not work as well for reaching the anterior cerebral artery or various posterior intracranial arteries.

Assuming effective ultrasound delivery is achieved, in addition to enhancing treatment of acute thrombotic or embolic ischemic stroke, TCD may also enhance and/or facilitate treatment of other cerebral disorders. For example, recurrent lacunar strokes, dementia, head trauma patients with intracerebral blood clots or perfusion abnormalities, and even Alzheimer's patients may benefit from TCD. In any such disorders, administration of TCD may help restore normal blood flow to the brain, help disperse harmful blood clots inside or outside blood vessels, and/or cause hyper-perfusion in one or more areas of the brain, thus enhancing cerebral function. For example, ultrasound administration has been shown to enhance the production of nitric oxide in or nearby blood vessels, which may thus cause vasodilatation of nearby arteries and arterioles and enhance tissue perfusion. (See, for example, W. Steffen et al., “Catheter-Delivered High Intensity, Low Frequency Ultrasound Induces Vasodilation in Vivo,” European Heart Journal (1994) 15, 369-376.) In any such treatments, however, use of TCD faces the same challenges in that it is very difficult to deliver at safe and effective frequencies to desired locations in the brain and thus can be performed only by a small handful of highly skilled technicians and can be directed only to a few areas in the brain. Also, the high intensities required to transmit ultrasound through the skull in TCD make its utility for treating any chronic disorder impractical, since any implantable power source used with a chronic, implantable ultrasound delivery device would be depleted too quickly.

Therefore, it would be desirable to have improved methods and apparatus for intracranial delivery of ultrasound energy for diagnostic ultrasound, therapeutic ultrasound, or both. Ideally, such techniques would be usable by a larger number of medical professionals than are currently qualified to administer TCD. Also ideally, such techniques would use ultrasound frequencies that do not cause unwanted bleeding in other blood vessels in the brain and that do not cause overheating or burning of the skin. At least some of these objectives will be met by the present invention.

### BRIEF SUMMARY OF THE INVENTION

Methods and apparatus of the present invention generally involve delivering ultrasound energy to a patient's intracranial space for diagnostic purposes, or therapeutic treatment, or both. The methods involve placing at least one access device on the scalp, skull, or partially through a hole in the skull, advancing at least one ultrasound delivery device partially through the access device, and transmitting ultrasound energy from the ultrasound delivery device(s) to the patient's intracranial space. When a hole in the skull is formed, the ultrasound delivery device can be placed over the hole, or partially through the hole, or at the edge of the skull, and then used to transmit energy to the patient's intracranial space.

In some instances, such as in the treatment of ischemic stroke, ultrasound energy is delivered to a target clot in a blood vessel. In other cases, such as in acute head trauma, ultrasound energy may be directed toward an extravascular blood clot in the brain. In other cases, energy may be delivered towards an area of blood vessels to cause vasodilatation and thus increase blood flow. Thus, the techniques and apparatus described herein may be used for a number of different applications and treatments.

Further aspects and embodiments of the present invention are described in greater detail below, with reference to the attached drawing figures.

### BRIEF DESCRIPTION OF THE DRAWINGS

FIG. 1 is an expanded sectional view of a portion of a human skull, showing a cross section of the skull, duramater, skull surface, epidural space, an access device with an introducer device and an ultrasound device together in place, in accordance with one embodiment of the present invention.

FIG. 2 is a perspective view of the ball used to modify the orientation of the ultrasound device or the introducer to the patient's skull, according to the present invention.

FIG. 3a is a top view of the access device of FIG. 1.

FIG. 3b is a cross-sectional view of the access device with a thin film interface mounted to the skull with an acoustically conductive patient interface located within the burr hole according to the present invention.

FIG. 3c is a cross-sectional view of the access device mounted to the skull with an acoustically conductive patient interface located within the burr hole and an additional patient interface located between the introducer and the patient according to the present invention.

FIG. 3d is a cross-sectional view of an alternative to FIGS. 3b and 3c, where an acoustically conductive material is located within the burr hole between the introducer and the patient.

FIG. 4a illustrates the thin film that is positioned between the access device and the skull surface according to the present invention.

FIG. 4b is a cross-sectional view of the elements shown in FIG. 4a, and includes a conductive pack integrated into the bottom side of the film.

FIG. 5a is a perspective view showing the ultrasound device of the present invention with an attached acoustically conductive patient interface inserted through the introducer.

FIG. 5b is a perspective view showing an ultrasound device of the present invention inserted through the introducer having an acoustically attached conductive material placed at the end of the ultrasound device inside the introducer, and an additional conductive material provided at the end of the introducer.

FIG. 6 shows an acoustically conductive media being attached to the ultrasound device within the introducer.

FIG. 7 is a cross-sectional view of a human skull and brain, showing the access device, the tubular introducer and the ultrasound device advanced in the skull and into a ventricle of the brain according to the present invention.

FIG. 8a is a cross-sectional view of a human skull, and brain, showing the access device and the ultrasound device directed to treat one portion of the clotted cerebral artery.

FIG. 8b is a similar view as FIG. 8a, showing the access device and the ultrasound device redirected to treat the second portion of the clotted cerebral artery.

FIG. 9 is a side view of a human head with three ultrasound devices coupled thereto, illustrating a triangulation technique for delivering ultrasound energy to a location in the brain, according to one embodiment of the present invention.

### DETAILED DESCRIPTION OF THE INVENTION

In one aspect of the present invention, a method for delivering ultrasound energy to a patient's intracranial space involves fixing at least one access device to the patient's skull, advancing at least one ultrasound delivery device at least partway through the access device, and transmitting ultrasound energy from the ultrasound delivery device to the patient's intracranial space. The access device may be fixed in place with screws through the scalp and into the skull, or alternatively the scalp may be retracted so that the base of the access device is located directly on the skull. If the scalp is retracted, it may be advantageous to also drill a hole through the skull near the center location of the access device to provide a route for minimal attenuation of the signals delivered from the ultrasound device to the intracranial space. In some embodiments, one hole is placed in the skull, and one ultrasound delivery device is used. In alternative embodiments, multiple holes are formed in the skull, and at least one ultrasound delivery device is advanced through the access device and at least partway through each hole. In other alternative embodiments, one hole is formed in the skull, and multiple ultrasound delivery devices are advanced through the hole.

The hole (or holes) in the patient's skull may be formed using any suitable devices and methods. For example, in some embodiments, a hand or power drill or burr device may be used, such as those commonly known in the art for forming holes in the skull. Once a hole is formed in the skull, one or more ultrasound delivery devices may be placed above the hole, advanced partway or completely into the hole or through the hole. In one embodiment, for example, an ultrasound delivery device is placed above the hole, at the edge of the skull or into the hole so that a distal end of the device is inserted through the access device and into the skull. In other alternative embodiments, one or more ultrasound delivery devices are advanced through the hole(s) into the epidural space, one or more ventricles and/or an intracerebral space of the patient's brain. For the purposes of this application, “intracerebral space” means any location within brain tissue or parenchyma outside of blood vessels.

To facilitate the introduction of ultrasound delivery devices through one or more holes in the patient's skull, one or more introducer devices may optionally be used. For example, in one embodiment, an introducer device is placed through the access device and at least partway into a hole, and at least one ultrasound delivery device is advanced partway or all the way through the introducer device. In one alternative embodiment, the introducer device is advanced through the access device and through a hole and into the patient's epidural space, and one or more ultrasound devices are advanced into the epidural space. In other alternative embodiments, the introducer device may be advanced through the hole and into a ventricle or an intracerebral space of the patient's brain, and one or more ultrasound devices are then advanced into the ventricle or intracerebral space. The introducer may be made of polymer or metal or of a composite construction.

To support the placement of the ultrasound device, either with the introducer or without the introducer, an access device may be used. The access device also has attributes that enable precise positioning and immobilization of the ultrasound device at a specific angle or range or angles with respect to the base or the access device and/or the skull. The access device can be a part of a stereotaxis frame, or it can be frameless and therefore directly secured to the skull. Examples of such frameless devices include the “Navigus System for Frameless Access” and the “Navigation” products made by Image-Guided Neurologics, Inc., located in Melbourne, Fla. Using either a stereotaxis frame or a frameless access device, the ultrasound device (with the introducer or without the introducer) may be placed on the scalp surface, on the skull surface, inside the skull, or positioned above the skull. The ultrasound device may also be directed to the treatment area and immobilized at a desired angle, thereby allowing longer therapy time without the risk of disengagement from the treatment target or misdirection by the ultrasound device. If the treatment area is of a larger size or length, the access device may allow re-positioning and can be used to immobilize the ultrasound device at various parts of the treatment area. For example, treatment of larger cerebrovascular clots may require that a proximal portion of the clot be targeted and treated first before repositioning the ultrasound device to target and treat a more distal portion of the clot. Alternatively, a large treatment area may be treated by either manually or automatically maneuvering the ultrasound device position through a range of angles with respect to the skull surface. Automatic maneuvering can be achieved by limiting the ultrasound device to a range of angles and then continuously powering the device through various angles by a power driven element (such as a motor). For example, in order to treat a cerebral clot which occludes several centimeters of the blood vessel it may be necessary to have the ultrasound device oscillate over a range of angles to treat the elongated clot. The angle between the ultrasound device and skull or base of the access device can range from 1 to 179 degrees, and more typically between 45 to 135 degrees. The ultrasound device can be limited to a specific range of angles through a plate having a slot placed about the ultrasound device, or around the ultrasound device and introducer. The plate can be fixed with respect to the base of the access device. The orientation and length of the slot would dictate the range of angles the ultrasound device could oscillate through.

Any suitable ultrasound delivery device may be used in implementing various embodiments of the present invention. For example, in one embodiment, the device may include an ultrasound device transducer. In another embodiment, the device may include a transducer-tipped ultrasound catheter. In either case, the ultrasound transducers may be formed from piezoelectric crystal or from silicon-based ultrasonic transducer technology.

In many embodiments, the ultrasound energy is transmitted acutely, such as in treatment of ischemic stroke or acute head trauma. In alternative embodiments, the ultrasound energy may be transmitted chronically, such as in treatment of chronic brain perfusion disorders or stroke rehabilitation. In some cases, a device or part of a device may be implanted in the patient for chronic treatment. In various embodiments, any of a number of different conditions may be treated or ameliorated according to the methods of the invention. For example, the ultrasound energy may be transmitted to a blood clot, either within or outside of a blood vessel, to help disrupt the clot. In another embodiment, the energy may be transmitted to a blood vessel to treat atherosclerosis of the vessel. In other embodiments, the energy may be transmitted to one or more blood vessels in the brain to help treat any of a number of blood perfusion abnormalities.

Optionally, the method may further include providing one or more pharmacologic agents to the patient, in conjunction with the delivered ultrasound energy. Examples of such agents include, but are not limited to, tissue plasminogen activator and other blood clot reducing agents, such as rTPA, Urokinease, Streptase (Streptokinase) Actiase (Alteplase) and Desmoteplase. Other agents which may be used include antiplatelet agents such as aspirin, Plavix (clopidorgel) and Ticlid (Ticclopidine), and GP IIb/IIIa inhibitors, such as Reopro (abciximab), Aggrestat (Tirofiban) and Integrilin (eptifibatide). Such a pharmacologic agent may be delivered intravenously, arterially, via intramuscular injection, or orally. Alternative methods involve delivering microbubbles or nanobubbles into the patient's bloodstream, in conjunction with the delivered ultrasound energy. Such microbubbles may be delivered intravenously or arterially. In some embodiments, both microbubbles and a pharmacologic agent(s) may be delivered to the patient along with the ultrasound energy. In some embodiments, pharmaceutical agent(s) may be delivered first, followed with delivery of microbubbles along with ultrasound energy. In other embodiments, microbubbles may be delivered first, followed by the delivery of pharmaceutical agent(s) along with ultrasound energy. Microbubbles may also be administered to the patient in a mixture with a pharmaceutical agent(s). In one such mixture, the majority of the pharmaceutical agent(s) may be present outside of microbubbles. In another mixture, the majority of the pharmaceutical agent(s) may be attached to microbubbles. Microbubbles and pharmaceutical agent(s) may also be administered simultaneously, through such means as through a single syringe or through more than one syringe. Ultrasound energy can be delivered to the patient prior to, during, or after, the delivery of a pharmaceutical agent(s) or microbubbles or both.

In one embodiment, one or more access devices are located on the scalp, skull, or partially through the skull, and ultrasound energy may be transmitted from any of several locations about the head and in any number of different pulsing sequences. For example, in one embodiment, access devices are located in several locations on the head, and ultrasound energy is simultaneously transmitted from multiple ultrasound devices at multiple locations. Such a delivery pattern of ultrasound about the head may be advantageous, for example, in triangulating the ultrasound transmissions towards the same target. In an alternative embodiment, ultrasound energy is delivered sequentially from multiple delivery devices. In some cases, the ultrasound energy is transmitted from multiple delivery devices with the same frequency and intensity. Alternatively, the ultrasound energy may be transmitted from multiple delivery devices with different frequencies, different intensities and/or different modes. Ultrasound energy may be transmitted at any desired frequency, although in preferred embodiments the energy has a frequency between about 10 KHz and about 20 MHz, and more preferably between about 20 KHz and about 10 MHz. According to different embodiments, the ultrasound energy may be transmitted in continuous mode or pulse mode, or may be modulated.

At any point during or after advancement of the access device, introducer or ultrasound device, the location of the apparatus may be monitored via any suitable visualization tools. For example, radiographic, computed tomography (CT) or magnetic resonance imaging (MRI) technologies may be used to help facilitate placement of an ultrasound delivery device in a desired location. In some embodiments, radiographs, CT images and/or MRI images may be used before device placement to determine an ideal location for the device. In some embodiments, during ultrasound energy delivery to the target site in the brain, patient recovery status may be monitored using a diagnostic ultrasound or one or more sensing methods, such as but not limited to the monitoring of oxygen levels or saturation, the rate of carbon dioxide production, heart rate, intracranial pressure and/or blood pressure. Also, the sensing element's measurements can be used to modulate the intensity, frequency and/or duty cycle of the ultrasonic device(s). Such a feedback process is also known as a closed loop control system.

Another aspect of the present invention includes the provision of a sterile or non-sterile acoustically conductive medium to facilitate ultrasound energy transmission to the targeted site. The acoustically conductive medium is positioned between the ultrasound transducer and the patient. The acoustically conductive medium may include a condense gel, diluted gel, oil, saline or any other semi-solid, fluid or gaseous material that conducts ultrasonic energy. The acoustically conductive medium may also be embodied in the form of a compliant pack which contains any of the above-identified acoustically conductive media inside the pack. In one embodiment, the pack has a thin conductive shell designed to contain the acoustically conductive medium. The compliant pack may be located within the hole in the skull, on the skull surface, on the scalp surface, at the tip of the introducer, at the tip of the ultrasound device, or inside or under the access device. The acoustically conductive medium may be delivered through the transducer or around the transducer, through the introducer or around the introducer, or through the access devices intermittently or continuously during the procedure. Low viscosity fluids may be preferred for this approach and may also assist in cooling of the ultrasound device and/or adjacent tissues (such as the scalp, skull or brain). Also, the acoustically conductive medium may be located within the hole in the skull, on the skull surface, on the scalp surface, as well as inside the access device and/or inside the introducer device.

In another aspect of the present invention, the access device has a base which includes a distal part and a proximal part, with a positioning movable member located between the distal and proximal parts. The distal part is attached to the skull and has a nest to receive a movable positioning member. The proximal part is attached to the distal part of the base and also has a nest to receive the movable positioning member. The proximal part of the base has a stabilizing element. The access device has a longitudinal aperture to receive the ultrasound device or other surgical or diagnostic tool therein. The distal part of the base is operable to be placed and affixed to the skull. In one embodiment, the positioning member includes a ball with a through-hole. The ball is movable within the nest between the distal and proximal parts of the base. The distal and proximal parts of the base may be attached using conventional methods such as bonding, frictional interface, fusing, welding, or screw(s). In one embodiment, the positioning ball can be made of a rigid material, and the size of its through-hole can be provided to match the size of the ultrasound device or other surgical tool to be used. In another embodiment, the positioning ball can be made of elastic material so that when the distal and proximal part of the base are tightened together, the size of the through-hole of the positioning ball is also tightened to immobilize the ultrasound device or other surgical tool received therein. In one embodiment, the stabilizing member includes at least two screws which are placed in the proximal part of the base, and which function to grip the ultrasound device, the introducer device or other surgical instruments at a fixed position and at a specific angle, as determined by the positioning ball.

In another aspect of the present invention, a thin film or a liner can be positioned between the access device and the skull, and/or between the ultrasound device and the skull. The film serves as a sterility barrier between the patient's inner tissue (epidural space) and the access device or the ultrasound device. The film can also serve as an acoustically conductive medium to facilitate ultrasound energy transmission. Also, the film may aid in the sealing of the burr hole to prevent bleeding of the skull. The film may have thrombogenic properties on its surfaces to enhance thrombosis of the scalp and/or skull bleeding. The film may be attached to the scalp, the skull, the access device, the introducer or the transducer device. The film can be composed of organic or synthetic polymers. The polymer material can be coated or impregnated with oil, gels, saline or other fluids to enhance its acoustically conductive properties. Alternatively, the film surfaces can be hydrophilic, thereby attracting fluid and/or ions that would also enhance its conductive properties.

In another aspect of the present invention, a method for delivering ultrasound energy to the patient's intracranial space involves positioning the ultrasound device at least partially through the access device and either locating the device juxtaposition to the scalp, skull or through a hole in the skull, at the edge of the hole in the skull or above the hole in the skull, and positioning the ultrasound device at the treatment target and immobilizing the ultrasound device. The ultrasound device may perform diagnostic functions, therapeutic functions or both functions.

According to another embodiment, the introducer may be placed between the access device and the ultrasound device. The introducer may be advanced through the access device and the ultrasound device may be placed through the introducer. A treatment target may be identified and located using diagnostic ultrasound techniques. Then, the introducer may be immobilized at the target treatment direction and exchanged for the therapeutic device. Ultrasound energy is then transmitted to the localized target in the patient intracranial space.

According to another embodiment, the introducer device may be placed through an access device located about a hole in the skull, and the ultrasound device may be advanced through an introducer and positioned in the patient's epidural space, intracerebral space or patient's ventricle, and ultrasound energy is transmitted into the patient intracranial space. A diagnostic ultrasound device may be used to locate the treatment side and a second ultrasound device may be used for therapy, or one ultrasound device may be used for both diagnostics and therapy. Such a method may further involve forming the hole(s) in the patient's skull.

Delivery of ultrasound energy through the scalp, the skull, a hole in the skull to the patient intracranial space either from above the hole or within the hole, from the epidural space, from the intracerebral space, or from the patient's ventricle, may be used for treatment of any of a number of conditions, such as acute clot outside of blood vessels caused by brain trauma, or ischemic stroke caused by a clot within a vessel. In various embodiments, ultrasound may be combined with delivery of a pharmacological agent(s) and microbubbles/nanobubbles. Ultrasound, with or without additional agents, may be delivered until the patient's symptoms improve and/or until a brain imaging study (e.g. MR, CT, PET, SPECT) demonstrate that the adverse “mass effects” of a clot are significantly reduced (e.g., <10% in size). For treatment of clot inside a vessel, as in ischemic stroke patients, the ultrasound delivery device may be placed near or directly adjacent to the clotted blood vessel.

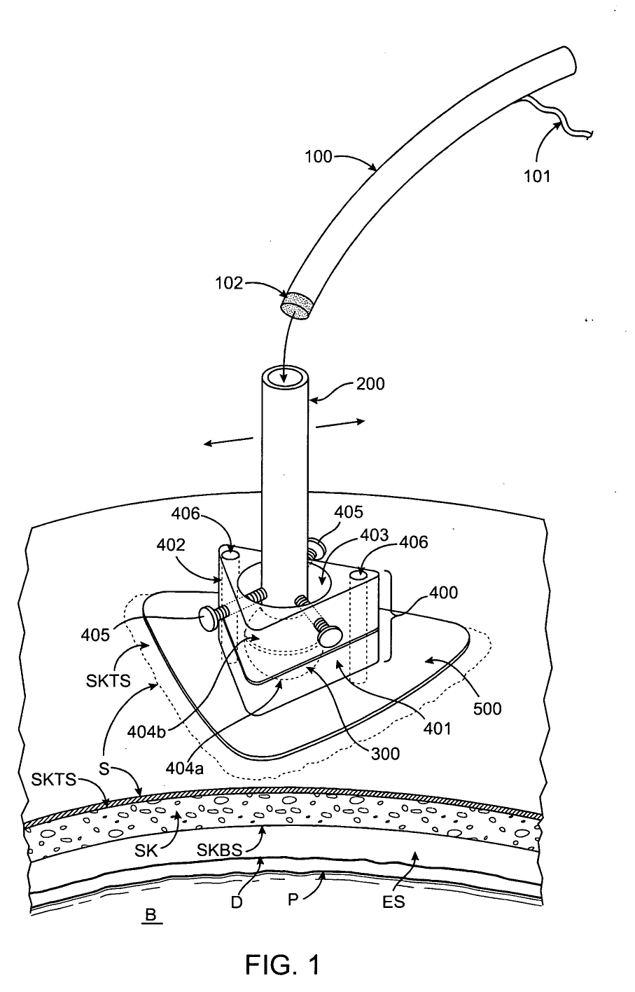

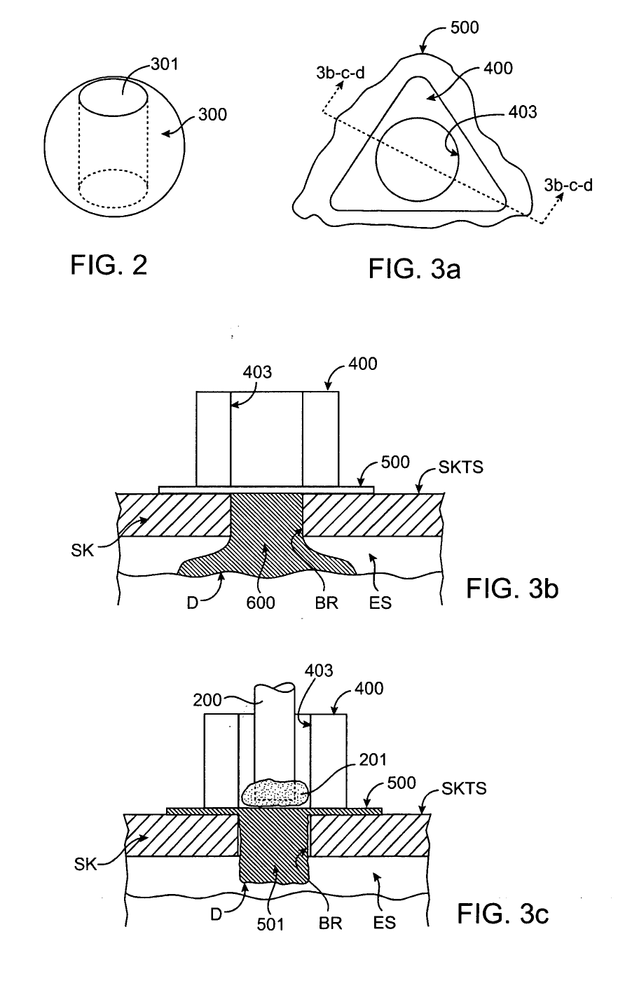

FIG. 1 is a cross-sectional view of a portion of a human head showing with an ultrasound assembly attached to the skull, and identifying the skull Sk, the skull's top surface SKTS, the skull's bottom surface SKBS, the scalp S, the pia matter P, the dura matter D, the epidural space ES, and the brain tissue B. An access device 400 is attached to the skull through the use of screw(s), pin(s) or adhesives (not shown). Through holes 406 can be used to enable the fixation of the access device 400 to the skull. The access device 400 can be used for placing an ultrasound device 100 or other surgical tools through the skull S with the ability to adjust the trajectory path of the ultrasound device 100 or surgical tool. The ultrasound device 100 can include electrical cables 101, and a conductive medium 102 provided at its distal end. The access device 400 has a base which includes a distal part 401 and a proximal part 402. Each part 401 and 402 defines a nest 404a and 404b respectively, therebetween that is size and configured to receive a movable positioning member 300. The access device 400 has a longitudinal aperture 403 which receives the introducer 200, the ultrasound device 100 or other surgical or diagnostic tools. The positioning member 300 of the access device 400 is positioned between the distal part 401 of the access device 400 and the proximal part 402 of the access device 400. The positioning member 300 has a ball with a through-hole 301 extending therethrough, as best shown in FIG. 2. The ball 300 is movable within the nest 404a and 404b between the distal part 401 and the proximal part 402. The distal part 401 of the access device 400 and the proximal part 402 of the access device 400 are attached together.

Stabilizing members 405 are provided within the proximal part 402 of the access device 400. The stabilizing members can be embodied in the form of at least two screws 405 which are placed in the proximal part 402 of the access device 400, and which function to grip the ultrasound device 100, introducer 200 or other surgical instruments, at a fixed position and at a specific angle, as determined by the positioning ball 300. The positioning ball 300 can be made of a rigid material and its through-hole 301 can be sized and configured to match the size of the introducer 200, the ultrasound device 100 or other surgical tool. Also, the positioning ball 300 can be made of elastic material so that when the distal part 401 and the proximal part 402 of the base 400 are tightened together, the size of the through-hole 301 of the positioning ball 300 can also be tightened, thereby immobilizing the ultrasound device or other surgical tool received therein.

The ball 300 enables the introducer 200 to be adjusted to a variety of angles with respect to the skull. Once the desired introducer angle or range of angles are identified by the user, the introducer 200 that is positioned through the pivoting ball 300 can be immobilized using the stabilizing members 405. Alternatively, the positioning ball does not need to be a separate element, but instead can be integrated with the distal end of the introducer 200 or the ultrasound device 100.

Also shown in FIG. 1, an acoustically conductive film 500 can optionally be positioned between the access device 400 and the skull prior to fixation of the access device 400 to the skull. A portion of the film 500 can be located on the skull's top surface SKTS, or alternatively the film 500 can be located on top of the scalp S (not shown).

To deliver ultrasound energy to the targeted intracranial site, the ultrasound device 100 can be delivered through the introducer 200 as shown in FIG. 1, or alternatively, the ultrasound device 100 can be used with the access device 400 without the introducer 200. The access device 400 is used to support the placement of the ultrasound device 100 in the skull, either with the introducer 200 or without the introducer 200. The access device 400 may be fixed in place with screws inserted through the scalp S and into the skull SK, or alternatively the scalp S may be retracted so that the distal part 401 of the access device 400 is positioned directly on the skull's top surface SKTS. If the scalp S is retracted, it may be advantageous to also drill a burr hole BR through the skull S, near the center location of the access device 400, to minimize related attenuation of the ultrasonic signals associated with transmitting through bone. To secure the film 500 in place, the same screws located in the holes 406 that are used to fix the access device 400 to the skull S can also be passed through the film 500. The screws can be coated, impregnated, covered, or constructed of a substance that minimizes bleeding at the entry sites of the screws. Alternatively, the screws can expand in situ to aid in hemostasis of the entry site.

The access device 400 may be a part of a stereotaxis frame, or it may be frameless and therefore directly secured to the skull. Using a stereotaxis frame or a frameless access device, the ultrasound device 100 (with the introducer 200 or without the introducer 200) may be placed on the scalp surface S, on the skull's top surface SKTS, inside the skull S, or positioned above the skull S. It may be directed to the treatment area and immobilized at a desired angle, thereby allowing longer therapy time without the risk of disengagement from the treatment target or misdirection by the ultrasound device.

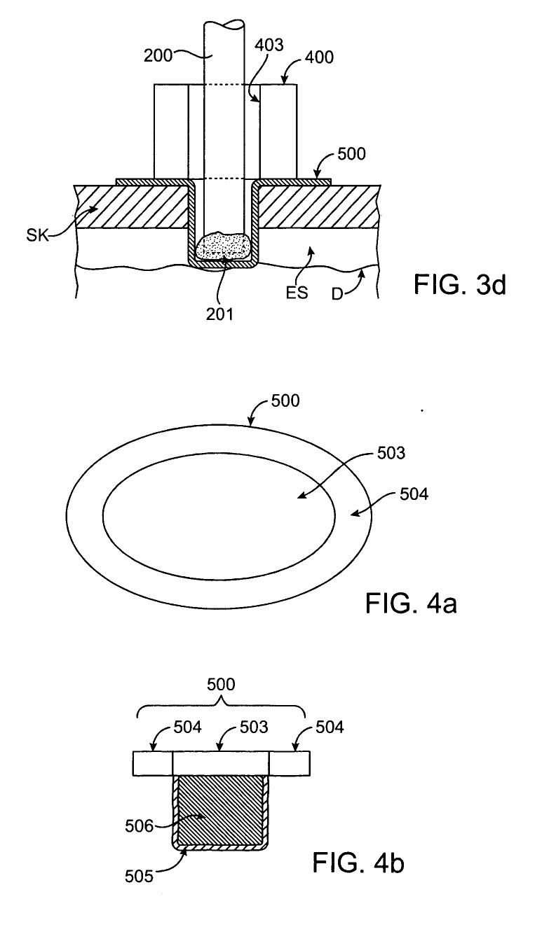

FIGS. 3a, 3b, 3c and 3d illustrate an access device 400 positioned on top of a hole in the skull. FIG. 3a is a top view of an access device 400 showing an aperture 403 and an acoustically conductive film 500 located under access device 400. FIG. 3b shows a cross section of FIG. 3a with a burr hole BR drilled through the skull SK, and with an access device 400 and a film 500 provided on the skull's top surface SKTS and about the burr hole BR. Acoustically conductive medium 600 can be provided in the burr hole BR. This acoustically conductive medium 600 can be sterile or not sterile, and is intended to facilitate ultrasound energy transmission from the ultrasound transducer 100 to the patient.

The acoustically conductive medium according to the present invention may include a condense gel, diluted gel, oil, saline or any other semi-solid, fluid or gaseous material that conducts ultrasonic energy. The acoustically conductive medium 600 may also be embodied in the form of a compliant pack which contains a gel, oil, saline or other acoustic conductive media contained inside the pack. The compliant pack can be positioned between the film 500 and dura mater D, but may also be located within the hole in the skull, on the skull's top surface SKTS, on the scalp surface S, at the tip of the introducer 200, at the tip of the ultrasound device 100 or inside or under the access device 400. Alternatively, the acoustically conductive medium 600 may be delivered through the ultrasound device 100 or around the ultrasound device 100, or through the introducer 200 or around the introducer 200, through the access device 400 either intermittently or continuously during the procedure. This media could also serve a second function by cooling the distal end of the ultrasound device and/or adjacent tissue.

FIG. 3c shows an alternative cross section of FIG. 3a. FIG. 3c provides a film 500 and an acoustically conductive media 600 that are integrated into a pack 501. Integrating these two elements into one member makes it easier for the user to assemble in situ. FIG. 3c also shows an alternative conductive media 201 located between the introducer 200 and the film 500. This alternative conductive media 201 enhances the transmission of ultrasound energy from the ultrasound device 100 to the patient's intracranial space.

FIG. 3d shows another alternative to FIG. 3b with the film 500 being flexible enough or pre-shaped to recess into or through the burr hole BR, and acoustically conductive media 201 being located between the introducer 200 and the flexible film 500. The thin film 500 or a liner may be located between the access device 400 and the skull S, or between the ultrasound device 100 and the skull S. The film 500 can serve as a sterility barrier between the epidural space or skull, and the access device 400 or the ultrasound device 100. The film 500 can also serve as an acoustically conductive medium to facilitate ultrasound energy transmission. Also, the film 500 can provide a sealing of the burr hole BR to prevent bleeding of the skull or scalp. The film surface can be provided with thrombogenic properties to enhance thrombosis of bleeding at the scalp S or the skull's top surface SKTS. The film 500 may be attached to the scalp S, the skull SK, the access device 400, the introducer 200 or the transducer device 100.

The film 500 can be composed of organic or synthetic polymers. The polymer material can be coated or impregnated with oil, gels, saline or other fluids to enhance its acoustically conductive properties. Alternatively, the film 500 can have hydrophilic properties so that upon attracting fluid and ions it would also enhance its conductive properties. The film 500 may be embodied in the form of one homogenous layer as shown in FIGS. 1, 3a and 3d, or it can be embodied as two sections, with an outer ring 504 and an inner ring 503 as shown in FIGS. 4a and 4b. The inner ring 503 is constructed of a material whose primary purpose is that of transmitting ultrasound energy. The outer ring 504 is constructed of a material whose primary purpose is helping promote hemostasis of any bleeding at the surgery site. Some examples of materials that can aid in promoting hemostasis include thrombin, cotton, adhesives, gel foam, cellulose, activated cellulose (such as thrombin impregnated or coated cellulose), Surgicel or Avitene. Alternatively, radio frequency energy and/or bipolar devices can be used to minimize bleeding about the surgical area. Also, bone wax could be used to line the burr hole BR to promote hemostasis. Referring also to FIG. 4b, the bottom surface of the film 500 can include a compliant ultrasonically conductive pack that can be constructed of a thin shell material 505 and a liquid or semi-liquid filler material 506. The material 506 can include materials such as oil, gels or saline.

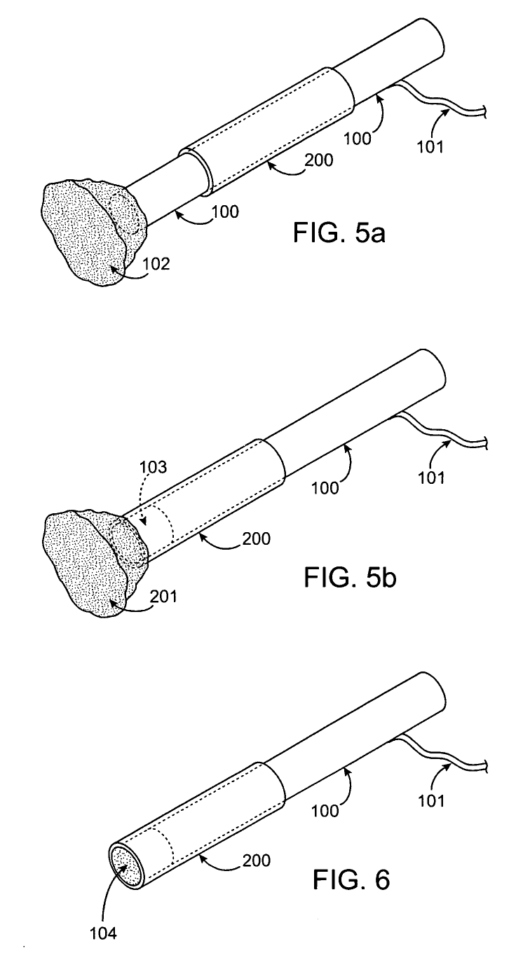

FIG. 5a shows ultrasonically conductive medium in form of a compliant pack 102 attached to the distal end of the ultrasound device 100. Alternatively, in FIG. 5b, a compliant pack 201 is attached to the distal end of the introducer 200 and additional conductive media 103 is provided inside the introducer 200 at the end of the ultrasound device 100.

FIG. 6 shows ultrasonically conductive medium in a different form of compliant pack 104 located inside the introducer 200 at the end of the ultrasound device 100.

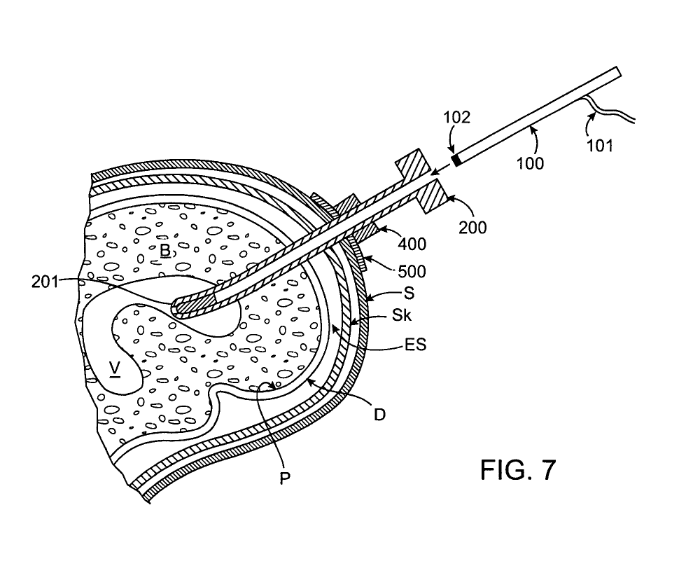

FIG. 7 shows a cross-sectional view of a human skull and brain. The access device 400 is placed on the skull, and the tubular introducer 200 (with acoustically conductive medium 201) is advanced in the skull and into a ventricle of the brain. The ultrasound device 100 (with acoustically conductive medium 102) may to be introduced through the introducer 200 and advanced into a ventricle of the brain. An acoustically conductive thin film 500 or a liner is provided between the access device 400 and the skull.

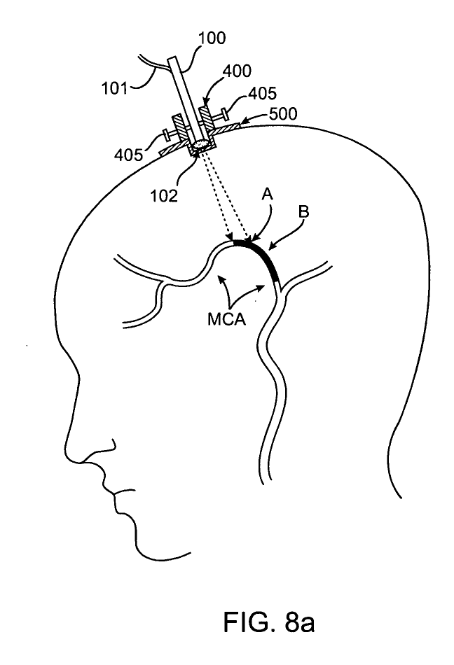

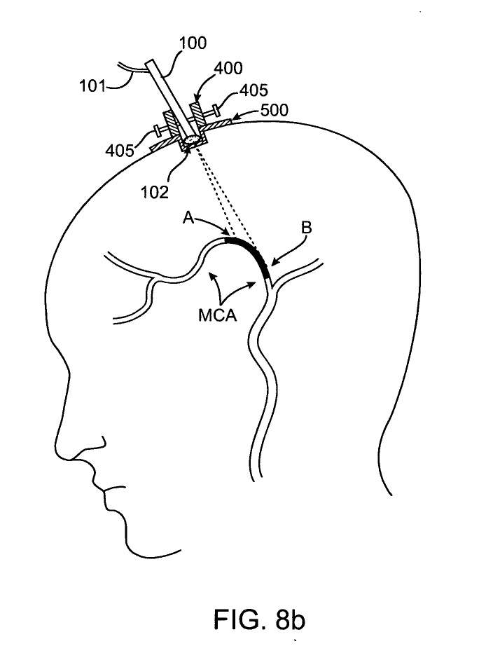

FIG. 8a is a cross-sectional view of a human skull, and brain, showing the access device 400 and the ultrasound device 100 targeting one portion of the clotted cerebral artery, treatment area A. Acoustically conductive medium 102 is positioned at the end of the ultrasound device 100 between the ultrasound device 100 and the patient. Stabilizing members 405 surround the ultrasound device 100 to immobilize the ultrasound device 100 with respect to the base of the access device 400 and with respect to the skull and the treatment area A.

FIG. 8b shows the access device 400 and the ultrasound device 100 of FIG. 8a redirected to treat a second portion of the clotted cerebral artery, treatment area B. Stabilizing members 405 are repositioned and immobilize the ultrasound device 100 within the access device 400 with respect to the skull and the treatment area B.

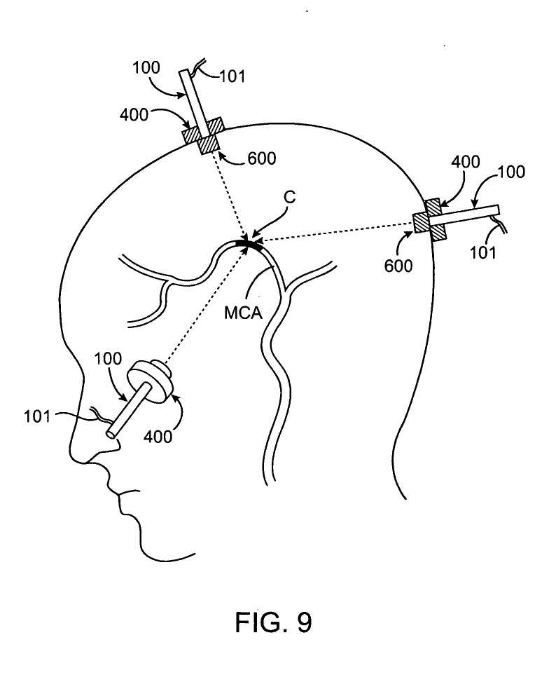

FIG. 9 shows the access devices 400 located in several locations on the head with ultrasound energy simultaneously transmitted from multiple ultrasound delivery devices 100 at multiple locations towards the treatment area C. Acoustically conductive medium 600 is located between each ultrasound device 100 and the patient. Such a delivery pattern may be advantageous, for example, in triangulating the ultrasound transmissions toward the same target to facilitate therapy process.

Although the invention has been described fully above, a number of variations and alterations could be made within the scope of the present invention. For example, in alternative embodiments, steps in the various described methods may be carried out in different orders or skipped altogether, and in other embodiments, additional optional steps may be added or one or more steps may be altered. Therefore, the foregoing description of exemplary embodiments should not be interpreted to limit the scope of the invention described by the following claims.

1. An apparatus for delivering ultrasound energy comprising:
an access device having an aperture;
an ultrasound device positioned partially through the access device, the ultrasound device having a distal end, and
an acoustically conductive medium juxtaposed at the distal end of the ultrasound device.

2. The apparatus of claim 1, further including a tubular introducer
positioned partially inside the access device, with the ultrasound device
positioned partially through the introducer.

3. The apparatus of claim 1, wherein the access device comprises a movable positioning member, a stabilizing member and a base.

4. The apparatus of claim 3, wherein the stabilizing member immobilizes the ultrasound device with respect to the base of the access device.

5. The apparatus of claim 3, wherein the stabilizing member limits the angulations of the ultrasound device with respect to the base of the access device.

6. The apparatus of claim 3, wherein the ultrasound device is actively moved within a range of angulations with respect to the base of the access device.

7. The apparatus of claim 3, wherein the movable positioning member adjusts the angulations of the ultrasound device with respect to the base of the access device.

8. The apparatus of claim 3, wherein the movable positioning member includes a ball having a through-hole.

9. The apparatus of claim 3, wherein the stabilizing member comprises of one or more screws.

10. The apparatus of claim 1, wherein the ultrasound device is adapted to allow delivery of acoustically conductive medium juxtaposition to the distal end of the ultrasound device.

11. The apparatus of claim 1, wherein the access device is adapted to allow delivery of acoustically conductive medium juxtaposition to the distal end of the ultrasound device.

12. The apparatus of claim 2, wherein the introducer is adapted to allow delivery of acoustically conductive medium juxtaposition to the distal end of the ultrasound device.

13. The apparatus of claim 1, wherein the acoustically conductive medium is partially in contact with the access device.

14. The apparatus of claim 1, wherein the acoustically conductive medium is partially in contact with the ultrasound device.

15. The apparatus of claim 2, wherein the acoustically conductive medium is partially in contact with the tubular introducer.

16. The apparatus of claim 1, wherein the acoustically conductive medium includes a fluid that conducts ultrasonic energy.

17. The apparatus of claim 1, wherein the acoustically conductive medium is retained inside a compliant pack.

18. The apparatus of claim 1, further including an acoustically conductive film which is partially in contact with the access device.

19. The apparatus of claim 18, wherein the acoustically conductive film further comprises an adhesive material that is attached to a surface of the film.

20. The apparatus of claim 18, wherein the acoustically conductive film further comprises a coating to inhibit infection.

21. The apparatus of claim 20, wherein the coating is selected from a group consisting of an anti-microbial coating and anti biotic coating.

22. The apparatus of claim 18, further comprising an acoustically conductive compliant pack in contact with a side of the film.

23. The apparatus of claim 3, further comprising a clot forming material juxtaposed to the base of the access device.

24. The apparatus of claim 1, wherein the ultrasound device is selected from the group consisting of an ultrasound transducer and a transducer-tipped ultrasound catheter.

25. The apparatus of claim 1, wherein the ultrasound device operates within the frequency range of 20 KHz to 10 MHz.

26. The apparatus of claim 1, wherein the ultrasound device operates in continuous mode.

27. The apparatus of claim 1, wherein the ultrasound device operates in pulse mode.

28. The apparatus of claim 1, wherein the ultrasound device operates in pulse mode and continuous mode.

29. The apparatus of claim 1, wherein the ultrasound device operates at a fixed frequency.

30. The apparatus of claim 1, wherein the ultrasound device operates at an adjustable frequency.

31. The apparatus of claim 1, wherein the ultrasound device operates at a modulated frequency.

32-71. (canceled)
